(19) United States
(12) Patent Application Publication
(10) Pub. No.: US 2007/0287897 A1
(43) Pub. Date: Dec. 13, 2007

(54) Optical Vascular Function Imaging System and Method for Detection and Diagnosis of Cancerous Tumors

(76) Inventor: Gregory Faris, Menlo Park, CA (US)

Correspondence Address: Lumen IPS, 2345 Yale Street, 2nd Floor, Palo Alto, CA, 94306, US

(21) Appl. No.: 10/586,824

(22) PCT Filed: Jan. 21, 2005
(86) PCT No.: PCT/US05/03090

§ 371 Date: Jul. 19, 2007

Related U.S. Application Data
(60) Provisional application No. 60/538,765, filed on Jan. 23, 2004.

(51) Int. Cl. A61K 49/00; A61B 5/06

(52) U.S. Cl. 600/310; 424/9.1

(57) ABSTRACT

An in vivo optical imaging system and method of identifying unusual vasculature associated with the angiogenic vasculature in tumors. An imaging system acquires images through the breast. Benign, noninvasive oxygen and carbon dioxide are used as vasoactive agents and administered by inhalation to stimulate vascular changes. Images taken before and during inhalation are subtracted. An optical vascular functional imaging system monitors abnormal vasculature through optical measurements on oxy- and deoxy-hemoglobin during inhalation of varying levels of O₂ and CO₂. The increase in contrast between tumor (cancerous) and normal (noncancerous) tissue is dramatic, facilitating accurate early detection of cancerous tumors and improving sensitivity and specificity (lower false negative and false positive rates). The invention is useful in mammography, dermatology, prostate imaging and other optically accessible areas.

### CROSS-REFERENCE TO RELATED APPLICATIONS

This application is a 371 filing claiming priority to PCT Application PCT/US05/03090 filed Jan. 21, 2005, which claims priority to U.S. Provisional Patent Application No. 60/538,765, filed Jan. 23, 2004, the entire content of which is incorporated herein by reference.

### STATEMENT REGARDING FEDERALLY SPONSORED RESEARCH OR DEVELOPMENT

This invention was made with Government support under Grant DAMD17-02-1-0570 awarded by the U.S. ARMY MEDICAL RSCH. ACQUISITION ACTIVITY. The Government has certain rights in this invention.

### BACKGROUND OF THE INVENTION

1. Field of the Invention

This invention relates generally to medical imaging systems and methods. More particularly, it relates to an innovative optical vascular functional imaging technology with significantly improved image quality, sensitivity and specificity, particularly useful in early detection and diagnosis of cancerous tumors such as breast cancer.

2. Description of the Related Art

Early detection is key to lower mortality rates associated with breast cancer. There is a continuing need for a better cancer screening system that can provide accurate early detection of breast cancer in a safe, noninvasive, relatively inexpensive manner. To lower the number of unnecessary biopsies, improved diagnosis tools are also highly desirable.

Currently, the standard screening modality for breast cancer is X-ray mammography. Unfortunately, X-ray mammography is less effective at detecting cancer in younger women's breasts, which are denser than those of older women. Moreover, although the risk of carcinogenesis resulting from X-ray mammography is relatively low, concerns about risks of exposure over many years of screening are valid. For these reasons, other imaging techniques are being used and studied to augment X-ray mammography, including ultrasound, MRI, Tc-99m sestamibi scintimammography, and PET. These imaging techniques are known in their respective fields and therefore are not further described herein for the sake of brevity.

Optical imaging techniques have also been explored. Optical imaging has many advantages, for instance, it is noninvasive, has no ionizing radiation, and requires no painful compression, etc. Optical mammography was closely studied in the 1970 and -1980s and proved to be inferior to X-ray mammography. The primary problem with optical mammography is its spatial resolution. Optical mammography has a spatial resolution of 0.5 to 1 cm, which means that blurring reduces contrast in smaller tumors.

U.S. Patent Application Publication No. 20050010114 by Porath, published on Jan. 13, 2005, entitled “OPTICAL MAMMOGRAPHY” attempts to address this problem by selectively imaging planes of the breast utilizing non-ionizing radiation. Porath's non-ionizing radiation imaging system uses a special contact window located between radiation detectors and tissue being imaged and a camera focused on a depth of a slice to be imaged.

Others have suggested administering, by injection or topological application, patients with contrast agents to reduce scattering. For example, U.S. Patent Application Publication No. 20030157021 by Klaveness et al., published on Aug. 21, 2003, entitled “LIGHT IMAGING CONTRAST AGENTS” proposes that contrast enhancement may be achieved in light imaging methods by introducing particulate materials as scattering contrast agents.

### BRIEF SUMMARY OF THE INVENTION

During the process of angiogenesis, tumors develop abnormal vasculature, and as a result, cancerous tissue is often hypoxic, a condition that can be observed with hemoglobin oxygenation measurements. The present invention utilizes the endogenous contrast afforded by the spectroscopic properties of hemoglobin together with exogenous vasoactive agents to improve detection of cancerous tumors with differential/dynamic optical imaging techniques.

We have discovered that inhalation of oxygen (O₂) and carbon dioxide (CO₂) can lead to significant contrast for in vivo optical imaging. Using O₂ and CO₂ as vasoactive agents to stimulate vascular changes has the additional advantage of being relatively safe, noninvasive, and requiring no injection or lengthy times between administration and imaging.

Using differential imaging with inspiratory contrast, our experimental results show that the additional contrast facilitates superior imaging quality than that of static (conventional) optical imaging. The increase in contrast between tumor (cancerous) and normal (noncancerous) tissue is dramatic. We have observed up to a factor of two variation in signal change. Taking advantage of this exogenous enhancement of the endogenous contrast due to oxy- and deoxyhemoglobin, the present invention provides clear contrasting images that would be particularly useful in early detection and diagnosis of cancerous tumors, potentially including breast cancer in women who are 40 or younger.

According to the invention, an imaging system acquires images through the breast. Images taken before and during inhalation of O₂ or CO₂ are subtracted. An enhanced optical vascular functional (physiological) imaging system monitors abnormal vasculature through optical measurements on oxy- and deoxy-hemoglobin during inhalation of varying levels of O₂ and CO₂. Where applicable, enhanced data analysis procedures are utilized to facilitate the image analysis on the large amount of data acquired. In an embodiment, a single optical imaging system monitors both static and dynamic contrast mechanisms, thus providing the best possible sensitivity and specificity.

Compared with what is achievable with the physical image information provided by x-rays, the present invention provides more specific functional image information particular useful for early detection and diagnosis of breast cancer. By detecting tumors generally missed on x-ray mammography (false negative results), the present invention can reduce the economic and human cost associated with later detection of disease. By reducing the number of false positive diagnoses, it could also reduce the worry and economic cost of unnecessary biopsies.

Furthermore, because of the low cost of optical instrumentation, the present invention could be used in combination with x-ray mammography, which should provide greater sensitivity and specificity than x-rays alone. With the transition to digital x-ray mammography, the present invention can even share the same camera with an x-ray imaging system, providing excellent registration of two different modalities.

Other objects and advantages of the present invention will become apparent to one skilled in the art upon reading and understanding the preferred embodiments described below with reference to the following drawings.

### BRIEF DESCRIPTION OF THE DRAWINGS

FIG. 1(a) is a schematic diagram of an immersion imaging system.

FIG. 1(b) is a schematic diagram of an immersion imaging system adapted for animals.

FIG. 2(a) is a static image of a mouse taken at 840 nm at 134 s after administration of carbogen.

FIG. 2(b) is the image from FIG. 2(a) with background subtracted.

FIG. 3 shows the temporal evolution of regions of the difference images at 780 nm.

FIG. 4 shows the temporal evolution of regions of the difference images at 840 nm.

FIG. 5 shows the temporal variation of relative changes in total hemoglobin (top), oxyhemoglobin (middle), and deoxyhemoglobin (bottom) during carbogen inhalation. The tumor region is shown by the dashed line; the region on the mouse torso away from tumor is shown by the solid line.

FIG. 6 shows the temporal variation of relative changes in total O₂ content (oxyhemoglobin change, minus deoxyhemoglobin change) during carbogen inhalation. The tumor region is shown by the dashed line; the region on the mouse torso away from tumor is shown by the solid line.

FIG. 7 shows relative concentrations of oxyhemoglobin (a) and deoxyhemoglobin (b) concentrations at 140 s (100 s after carbogen administration).

FIG. 8 shows normalized eigen value spectrum.

FIG. 9 shows first two eigen images from principal component analysis.

FIG. 10 shows the temporal variation of the eigen image scaling factor.

FIG. 11 illustrates imaging of a human subject with immersion of the breast.

FIG. 12 illustrates imaging of a human subject with immersion and mild compression.

FIG. 13 illustrates a form of 3-D data for differential vasoactive imaging.

### DETAILED DESCRIPTION OF THE INVENTION

A primary goal of the invention is to develop reliable and yet inexpensive technology to improve sensitivity and specificity (lower false-negative and false-positive rates) for early breast cancer detection and diagnosis. We have achieved this goal with enhanced functional (physiological) optical imaging using a new type of contrast based on the unusual vascular function of tumors (atypical oxygenation improvement, atypical vasoactivity, and blood pooling).

Another goal is to improve imaging through dense breasts where X-ray mammography is less successful. We have been investigating this differential vasoactive optical imaging (DVOI) approach in animal model studies. That work has demonstrated strong contrast between cancerous and noncancerous tissue during differential imaging in rodents in association with inhalation of O₂/CO₂ gas mixtures.

The contrast achieved by DVOI results from the vasculature in tumors and can arise from atypical oxygenation improvement, atypical vasoactivity, and blood pooling, as monitored by varying the levels of inspired O₂ and CO₂. These differential vascular function measurements can be used to augment the cancer-specific static contrast derived from 1) elevated hemoglobin concentrations from angiogenesis and 2) reduced local hemoglobin oxygenation from tumor hypoxia.

A single DVOI system can monitor both static and dynamic contrast mechanisms, thus providing the best possible sensitivity and specificity from an optical imaging system. CO₂ and O₂ are attractive contrast-enhancing agents because they are benign, safe at appropriate concentrations and inhalation periods and require no injection or lengthy times between administration and imaging.

Using these inspiratory contrast agents, we observed strong contrast between images taken before and during inhalation. We found that optical techniques can detect and locate picomole variations in chromophore concentrations over optical thicknesses comparable to those of the human breast. In the following sections, we describe how the specificity of the differential contrast available with the DVOI approach is sufficiently significant to allow tumor detection with higher sensitivity, even at the poor spatial resolution available using optical imaging through the human breast.

Advantages of using DVOI for breast imaging include functional imaging (i.e., imaging that provides information on tissue state and function), inexpensive instrumentation, and no ionizing radiation. DVOI could prove useful as a primary screening modality. Alternatively, it would be very useful as a secondary imaging modality to X-ray imaging for diagnosing, staging, or monitoring treatment of breast cancer. Because of its simplicity and low cost, DVOI can be efficiently incorporated into an X-ray or ultrasound imaging system to provide functional information to complement the physical imaging of these modalities. DVOI may prove more effective in imaging dense breasts and may reduce or avoid the unpleasant or even painful compression used for X-ray mammography.

Optical Breast Imaging

As discussed above, the primary problem with optical mammography is spatial resolution. Optical mammography has a spatial resolution of 0.5 to 1 cm, which means that blurring reduces contrast in smaller tumors. This limitation can be overcome by providing functional imaging information.

Functional Optical Imaging

Whereas X-ray imaging primarily provides structural information, optical spectroscopy imaging can provide information both on structure and tissue function. For example, optical measurements at different wavelengths can indicate total hemoglobin content and oxygenation—functional information that is significant for breast cancer detection. Tumor angiogenesis typically leads to elevated local hemoglobin concentrations. In addition, tumors are often hypoxic, which can be observed optically as a decrease in hemoglobin oxygenation. Because tumors that are more hypoxic tend to be resistant to radiotherapy and chemotherapy and are more likely to be metastatic or invasive, the degree of tumor hypoxia can be used to guide treatment.

Tumor morphology also provides a source of contrast through variations in the optical scattering coefficient. The inventive system augments functional optical imaging with differential measurements related to tumor vascular function, taking advantage of the full range of available optical contrast. The broadest use of available contrast is the most effective for improving sensitivity and specificity.

The atypical characteristics of vasculature produced through tumor angiogenesis provide the scientific basis for the differential vasoactive optical imaging approach disclosed herein. The following articles, incorporated herein by reference, disclose information related to tumor angiogenesis: J. M. Brown and A. J. Giaccia, “The unique physiology of solid tumors: Opportunities (and problems) for cancer therapy,” Cancer Res. 58, 1408-1416 (1998); and P. Carmeliet and R. K. Jain, “Angiogenesis in cancer and other diseases,” Nature 407, 249-257 (2000).

Blood vessels in tumors often exhibit distended capillaries with leaky walls and sluggish flow. These properties provide at least three types of contrast for optical imaging in conjunction with varying levels of inspired O₂ and CO₂. These types of contrasts are due to atypical oxygenation improvement, atypical vasoactivity, and blood pooling. Because both O₂ and CO₂ are vasoactive, atypical tumor vasoactivity arising from administration of changing levels of these gases should provide strong imaging contrast. Tumor vessels are often contorted and leaky; thus, blood pooling in these vessels will delay response to oxygenation changes, providing another good contrast mechanism. Blood pooling itself can contribute to the atypical oxygenation improvement in tumors. However, our experiments indicate that atypical oxygenation improvement persists beyond the transient response caused by blood pooling.

Using functional optical imaging, the DVOI system disclosed herein can reliably measure the unusual vasculature in tumors. For example, by comparing hemoglobin content before and after carbogen is administered, opposing vasodilation and vasoconstriction responses after 15% CO₂ and 85% O₂ (carbogen) inspiration are readily detectable. Similarly, the changing response in tumor oxygenation after increased O₂ administration is easily measured by monitoring hemoglobin oxygenation levels before and after the O₂ level is increased. Changes associated with blood pooling are observable in delayed oxygenation changes in the tumor. The DVOI approach could also incorporate quantitative measurements of oxy- and deoxy-hemoglobin to improve overall sensitivity and specificity.

DVOI very possibly can provide functional discrimination between benign and malignant lesions. Benign lesions tend to have rounded vasculature while malignant lesions tend to be more angular. Because the vasculature is different, it is likely that the vascular response to O₂ and CO₂ will also be different.

There are additional motivations for examining differential contrast such as that associated with tumor vascular function. First, because the breast is highly heterogeneous, comprising the lobes (glandular tissue), fat, connective tissue, ducts, and supporting vasculature, using a broader palette of contrast mechanisms should provide more specificity for optical imaging and help compensate for that heterogeneity. Second, the more successful noninvasive optical measurements (e.g., pulse oximetry, functional brain imaging) are differential or dynamic. Finally, recent theoretical work has demonstrated improved results using dynamic or differential optical imaging techniques, both of which rely on changes in optical contrast over time. In the following examples, we combine dynamic and functional measurements to obtain the best possible results.

### EXAMPLES

### Differential Vasoactive Optical Imaging System Setup for Animals

To monitor contrast for a range of tumor sizes and stages of development, we performed DVOI on and took noninvasive measurements from mice and rats. To replicate tissue thicknesses similar to those of the human breast, we partially immerse the anesthetized animals in liquid tissue phantoms that simulate the optical properties of human breast tissue. Although this approach does not allow for the effects of tissue heterogeneity in the breast, it is the most practical method for studying contrast without actually using human subjects. The measurements are noninvasive and thus can be readily repeated on animals as our instrumentation and methods are refined/optimized.

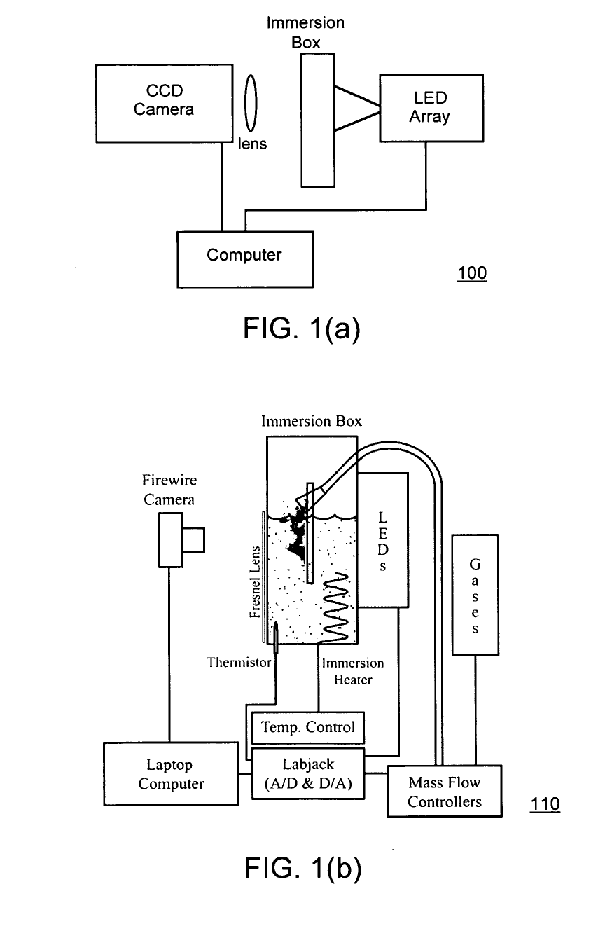

FIG. 1(a) shows a continuous wave (CW) immersion imaging system 100 for performing DVOI with immersion. The system 100 comprises a near-infrared (NIR) light source and a camera, both of which are connected to a computer capable of analyzing image data in substantially real time. An immersion container is positioned between the light source and the camera for holding the imaging subject. In some embodiments, the light source is made up of an array of bright light emitting diodes (LEDs) and the camera is a digital camera with high sensitivity and high SNR.

As one skilled in the art will appreciate, the system can be readily implemented in various ways. FIG. 1(b) shows an exemplary system 110 adapted for animal model studies. In a specific embodiment, these LEDs emit near infrared (NIR) radiation with peak intensities at either 780 or 840 nm (Epitex L780-01AU and Epitex 840-01KSB, respectively). Switching between LED arrays enables measurements at different wavelengths and the determination of hemoglobin content and hemoglobin oxygenation. We increased light throughput onto the imaging sensor by 20% by installing a large-aperture lens with high NIR transmission (JML Optics).

The NIR light source is directed at the sample immersion box, which contains the study animal in a heated (37° C.), matching medium composed of water, ink, and submicrometer polymer spheres (Ropaque from Rohm and Haas Company). This immersion medium approximates the scattering and absorptive properties of the mouse tissue. The front of the immersion box is imaged onto the camera. Images at each individual wavelength are then collected, digitized (8-bit resolution), and sent to the computer for analysis.

The DVOI system can be readily implemented with a variety of suitable cameras, for example, the Dragonfly CCD (charge-coupled device) camera (Point Grey Research), the Pulnix TM-9701 CCD camera coupled to a Stanford Photonics Gen III image intensifier, and the ImagingSource DMK-3002-IR. Preferably, the system employs the digital Dragonfly CCD camera because it offers a significant improvement in signal-to-noise ratio (SNR) over other video cameras. Although the Dragonfly has a lower absolute sensitivity in the NIR region compared with the other video cameras, it has lower read noise and is capable of longer exposure times (>60 s), which is important for imaging thicker tissue samples. More expensive cameras are available that provide superior sensitivity and sensitive area such the Retiga Exi manufactured by QImaging.

The compensation provided by immersing the animal (or at least the region of interest) in a tissue phantom improves image quality by removing changes in contrast associated with changes in tissue thickness and geometry, allowing better use of the dynamic range of the camera and providing more uniform illumination. When the match is good, the tissue almost disappears, and the image shows variations due to internal structure and contrast, which is what we want for in vivo imaging. The immersion medium serves to: (1) allow study of an effective tissue as thick as is typical for the human breast, and (2) enhance measurements by eliminating the effects of boundaries. Although the tissue phantom lacks the heterogeneity of the human breast, there is considerable heterogeneity in the animal itself.

Tissue phantoms are prepared using our established methods, which are disclosed in M. Gerken and G. W. Faris, “Frequency-domain immersion technique for accurate optical property measurements of turbid media,” Opt. Lett. 24, 1726-1728 (1999); and X. Wu, L. Stinger, and G. W. Faris, “Determination of tissue properties by immersion in a matched scattering fluid,” Proc. SPIE 2979, 300-306 (1997), both of which are incorporated herein by reference.

After an initial tissue phantom is prepared, an animal with a target region to be imaged is immersed between the source and collection fibers, the changes in amplitude and phase are measured, and the phantom composition is adjusted according to the optical properties determined from the immersion measurement. This process is repeated until the optical properties of the immersion medium and the imaged tissue agree to within a few percent. The thickness of the tissue phantoms is varied by inserting Plexiglas sheets into the box containing the tissue phantom for the CW measurements.

Animal Models

Human breast cancer cells (MDA 231) and mouse embryonic fibrosarcomas were grown in Dulbecco's minimum essential medium (DMEM) with glutamine and 10% fetal bovine serum. The cells were harvested when they were 80% confluent, using 0.25% trypsin. Cells were injected subcutaneously on the dorsum of the female athymic nude mice (approximately 23 g, Harlan Laboratories). Both cell lines were used at a concentration of 2-3 million cells in 100 μl of DMEM for each animal. The tumor volumes were measured twice weekly.

Animal Imaging

Imaging experiments were conducted on animals with tumor volumes of 500-1000 mm³. We used two-four animals for each experiment. After being anesthetized with 40 mg/kg of pentobarbital, the mice were secured to a 3-mm Plexiglas platform with black vinyl tape. Anesthesia was given in further doses of 20 mg/kg as needed to reduce stress associated with immersion and to keep the animal immobilized. Carbogen or air was administered to the immersed mouse via a nose cone at a flow rate of approximately 3 l/min. The optical path length of the immersion box was adjusted to match the thickness of the mouse (˜2-2.5 cm). At this thickness, the exposure time of the camera allowed us to measure both wavelengths at approximately three frames per second.

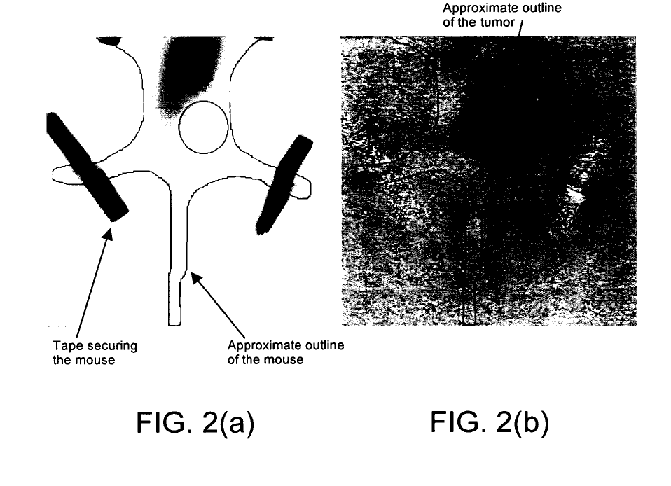

Images of individual mice were recorded before, during, and after the administration of carbogen. FIG. 2(a) shows one of these static images taken 134 s following the administration of the carbogen. The approximate outlines of both the mouse and the tumor have been placed on top of the image as a guide. The mouse's head is out of the immersion medium and is above the field of view. The hind legs and tail are seen at the bottom of the image. FIG. 2(b) shows this same image after the subtraction of a background, which is simply an image of the mouse before the carbogen was turned on. Although the boundaries of the mouse and tumor are obscured by the good match with the immersion medium, it is clear from the difference image in FIG. 2(b) that there are distinct regions of contrast between the tumor and the surrounding tissues of the mouse.

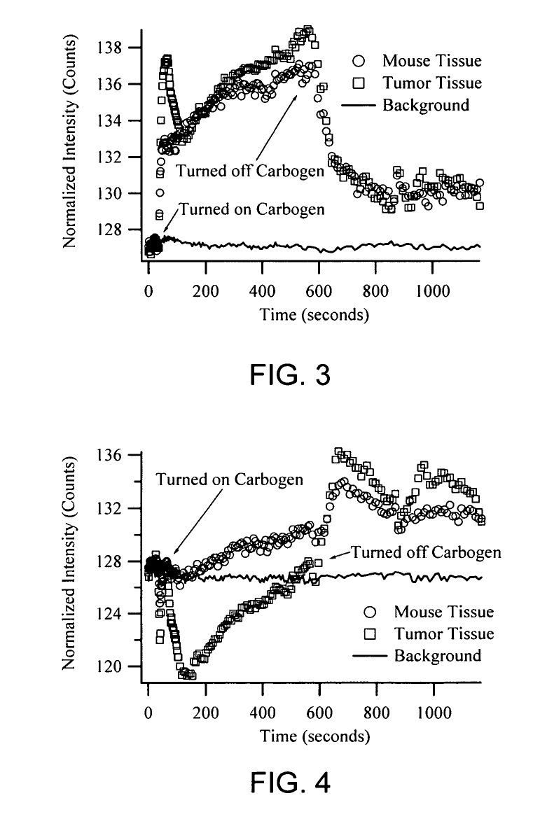

Temporal Variation in Differential Contrast The enhanced contrast between the tumor tissue and the mouse tissue due to the inhalation of the carbogen was monitored by averaging the changes in intensity over areas within the difference images. FIGS. 3 and 4 show these averaged data for differences in the 780 nm and 840 nm images, respectively. The squares represent changes in the tumor tissue, the circles indicate an adjacent region within the mouse that does not contain the tumor, and the line represents the average of a part of the image not containing the mouse.

The maximum change for both wavelengths is approximately ±10 units, and it is clear from the figures that distinct differences occur for the dynamics of the tumor tissue when compared with the normal mouse tissue. Furthermore, the background, which is a measure of lower limits for detection, varies just ±0.2 units.

FIGS. 3 and 4 indicate that several regions (e.g., near 55 s at 780 nm, and near 135 s at 840 nm) show strong contrast between tumor and surrounding tissue. Additional contrast is found after the carbogen is stopped; for 840 nm, the relative intensity of tumor and surrounding tissue reverses.

Although images at a single wavelength such as FIG. 2(b) can be useful for cancer detection, it is also of interest to determine the changes in oxyhemoglobin and deoxyhemoglobin. We have analyzed the same image data set used to produce FIGS. 3 and 4 to calculate approximate path-integrated oxyhemoglobin and deoxyhemoglobin. The absorption at 780 nm and 840 nm can be described as:
μₐ^{λ}=2.3{ε_{Hb}^{λ}[Hb]+ε_{HbO}_{₂}^{λ}[HbO₂]} (1)
where □ is the wavelength of interest, [Hb] and [HbO₂] are the concentrations (moles/L) of deoxygenated and oxygenated hemoglobin, respectively, and □ is the molar absorption coefficient. Using Beer's Law, we can describe the change in the absorption coefficient □ₐ, at time t after a baseline image has been taken as:

$$Δ {μ}_{a}^{λ} = {μ}_{a}^{λ , t} - {μ}_{a}^{λ , baseline} = 2.3 {log}_{10} [ \frac{{I}_{baseline}}{{I}_{t}} ] \text{/} l \qquad ( 2 )$$

where I is the intensity of transmitted light and l is the pathlength in cm, corrected appropriately for the differential pathlength factor for the animal tissue. We can obtain a rough measure of the change in path-integrated oxyhemoglobin and deoxyhemoglobin concentrations by assuming that the differential pathlength factor is the same at both wavelengths. By manipulating equations (1) and (2), we see that:

$$( \begin{matrix}Δ {μ}_{a}^{780} \\ Δ {μ}_{a}^{840}\end{matrix} ) = \frac{2.3}{l} * [ \begin{matrix}{ɛ}_{Hb}^{780} & {ɛ}_{{Hb}_{{o}_{2}}}^{780} \\ {ɛ}_{Hb}^{840} & {ɛ}_{{Hb}_{{o}_{2}}}^{840}\end{matrix} ] ( \begin{matrix}Δ [ Hb ] \\ Δ [ {HbO}_{2} ]\end{matrix} ) \qquad ( 3 )$$

Because of the finite bandwidth of the LEDs, we calculated the absorption coefficient by integrating the wavelength-dependent absorption coefficient with the normalized spectra of the LEDs for each wavelength respectively:
εⁱ=∫ε(λ)Iⁱ(λ)dλ (4)

This led to the following equations for the concentrations of Hb, HbO₂, Hbₜₒₜₐₗ at time t:

$$\begin{matrix}Δ [ Hb ] ( t ) = 2.3 * ( 7.507 * {10}^{- 4} * {log}_{10} \frac{{I}_{B}^{780}}{{I}_{t}^{780}} - 5.271 * {10}^{- 4} * {log}_{10} \frac{{I}_{B}^{840}}{{I}_{t}^{840}} ) \text{/} l , & ( 5 ) \\ Δ [ {HbO}_{2} ] ( t ) = 2.3 * ( - 5.225 * {10}^{- 4} * {log}_{10} \frac{{I}_{B}^{780}}{{I}_{t}^{780}} + 7.996 * {10}^{- 4} * {log}_{10} \frac{{I}_{B}^{840}}{{I}_{t}^{840}} ) \text{/} l , & ( 6 ) \\ Δ [ Hbtot ] ( t ) = Δ [ Hb ] ( t ) + Δ [ {HbO}_{2} ] ( t ) & ( 7 )\end{matrix}$$

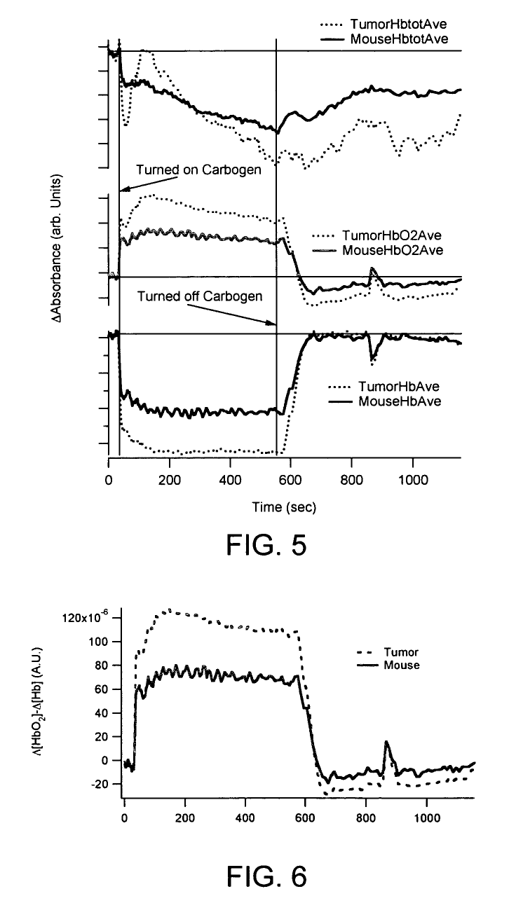

We used these calculations to determine the approximate temporal variation of the total hemoglobin, oxyhemoglobin, and deoxyhemoglobin shown in FIG. 5. These values were in turn used to calculate the approximate change in O₂ content (oxyhemoglobin change, minus deoxyhemoglobin change) shown in FIG. 6. Several observations arise from these images: The tumor vasculature shows more erratic behavior, as seen from the oscillations at the beginning of carbogen inhalation. The failure to return to baseline for the total hemoglobin concentration (FIG. 5), and the overshoot in O₂ content at the end of the carbogen inhalation (FIG. 6). The magnitude in changes of oxyhemoglobin and deoxyhemoglobin are accentuated in the tumor (FIG. 5). The increase in O₂ content of the tumor is delayed relative to the rest of the animal (FIG. 5 middle and FIG. 6), which may be due to blood pooling in the tumor.

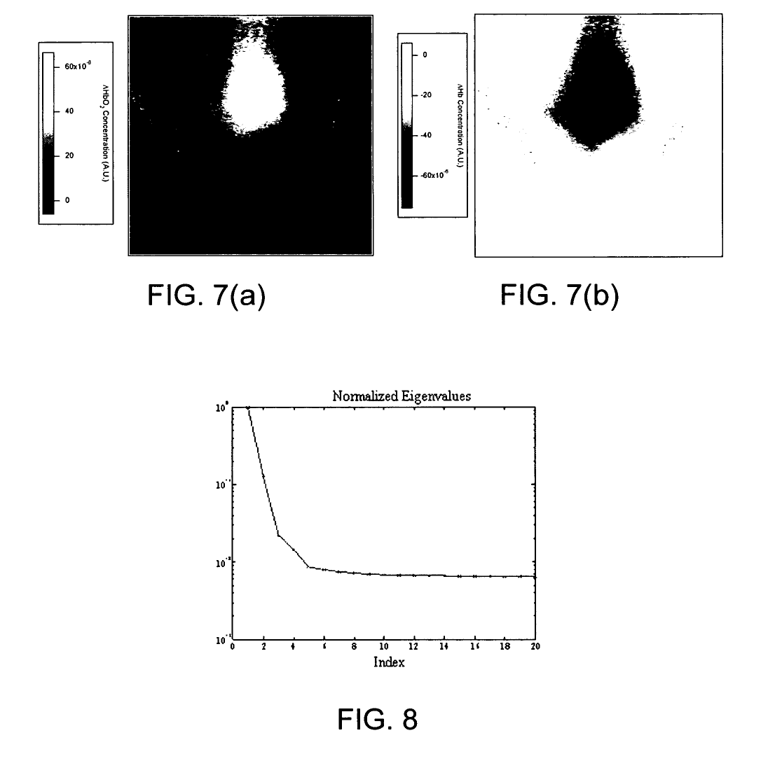

The same processing used for FIGS. 5 and 6 can be used to produce images representing approximate path-integrated oxyhemoglobin and deoxyhemoglobin as shown in FIGS. 7(a) and 7(b), respectively. These differential vasoactive images show a dramatic increase in tumor contrast as compared with a raw or static image, see, e.g., FIG. 2(a).

Principal Component Analysis

The imaging experiments described above generated large sets of data. Typically, images with 10⁵ pixels at two wavelengths are recorded every 2-10 seconds over the cycling period of carbogen administration (approximately 10 to 20 minutes). Based on these experimental results, we expect to see <7% change in image intensity following carbogen administration. Because extracting such small signal changes from large data sets poses a formidable challenge, researchers have developed techniques that generate smaller sets of orthogonal images to describe the generated data, see, e.g., incorporated herein by reference, L. Sirovich and E. Kaplan, “Analysis methods for optical imaging,” in Methods for In Vivo Optical Imaging of the Central Nervous System, R. Frostig, Ed. (CRC Press, 2001); and L. Sirovich and R. Everson, “Management and analysis of large scientific datasets,” Intl. J. Supercomputer Applications 6, 50-68 (1992). In practice, these methods have been shown to accurately describe data sets of 10,000 images with only ˜100 eigen images.

In the most basic adaptation of these methods, known as principal component analysis (PCA), the set of recorded images is represented by:
f=f(t,x) (8)
where x describes the spatial pixel grayscale values of the image, and t is the time at which the image data was collected. Researchers have shown that these images, f(t,x), can be decomposed into the set of orthogonal functions aₙ(t) and □ₙ(x) by:

$$f ( t , x ) = {\sum }_{n}^{} {μ}_{n} {a}_{n} ( t ) {φ}_{n} ( x ) . \qquad ( 9 )$$

A series of T time images containing P pixels can be described by the matrix:

$$M = [ \begin{matrix}f ( 1 , 1 ) & f ( 1 , 2 ) & … & f ( 1 , P ) \\ f ( 2 , 1 ) & f ( 2 , 2 ) & … & f ( 2 , P ) \\ ⋮ & & & ⋮ \\ f ( T , 1 ) & … & … & f ( T , P )\end{matrix} ] \qquad ( 10 )$$

This matrix can then be decomposed into the different aₙ(t) and □ₙ(x) components through the general technique of singular value decomposition:

$$\begin{matrix}{A}_{n} = [ \begin{matrix}{a}_{n} ( 1 ) \\ ⋮ \\ {a}_{n} ( T )\end{matrix} ] , {V}_{n} = [ \begin{matrix}{φ}_{n} ( 1 ) \\ ⋮ \\ {φ}_{n} ( P )\end{matrix} ] , and U = [ \begin{matrix}{μ}_{1} & & 0 \\ & ⋰ & \\ 0 & & {μ}_{T}\end{matrix} ] \\ and & ( 11 ) \\ M = {AUV}^{†} & ( 12 )\end{matrix}$$

The columns of V contain the orthonormal spatial basis functions, the orthonormal columns of A describe the time-dependence of the spatial basis functions, and U contains the weighting factors for the two matrixes A and V.

As a first step in processing the data, we apply this simplified PCA method to determine changes in oxyhemoglobin and deoxyhemoglobin, scaled by some pathlength factor l as described above. The time-dependent images that describe □[Hb] and □[HbO₂] were ordered into a matrix as shown in equation (10), and the singular value decomposition was carried out to obtain the matrices A, U, and V. FIG. 8 presents a plot of the normalized scaling factors contained along the diagonal of U. Only the first three or four eigen images contribute significantly to the set of images that describe the hemoglobin dynamics in our study.

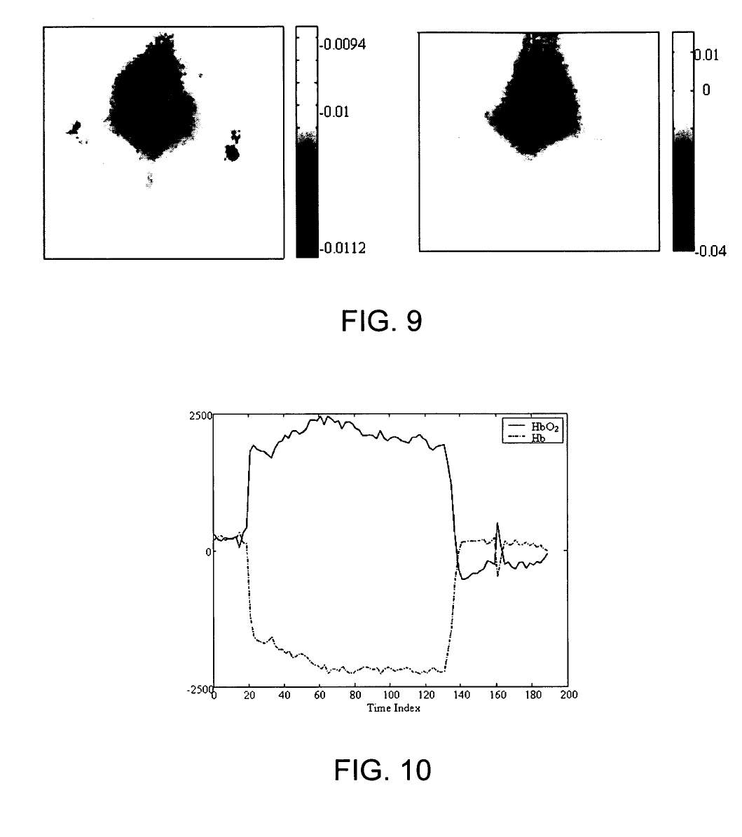

FIG. 9 shows the first two eigen images corresponding to the first two columns of matrix V. The contrast between the tumor and the surrounding tissue is evident in the second image. The time-dependent weighting of the second eigen image in the □[Hb](t) and □[HbO₂](t) sets of images can be determined from the matrix product of A·U, and is shown in FIG. 10.

Differential Vasoactive Optical Imaging System Setup for Humans

DVOI is very effective for breast cancer detection, and is preferred for screening young women with known propensity for developing breast cancer. Combined with another imaging modality such as x-ray imaging, the DVOI system can prove to be a powerful tool in combating the disease.

In the case of human subjects, different imaging methods may be used for differential vasoactive imaging of the breast. The imaging may be performed with or without compression and with or without immersion. In some cases, optimal imaging entails using at least mild compression and immersion.

Mild compression is advantageous for two reasons: first, with compression the total imaging distance is less, leading to a higher SNR, and hence increasing the likelihood of detecting a smaller tumor. Second, X-ray mammography uses compression. The combination of optical imaging with X-ray imaging provides a further embodiment of the invention—given the low-cost of X-ray imaging and the possibility that both imaging techniques could be performed simultaneously. In a still further embodiment, both imaging systems share the same detector in the case where digital mammography is used via semiconductor-based cameras. That combination would lead to an improvement in sensitivity and specificity over either modality alone. This embodiment requires coregistration of images from the two modalities, which could be achieved most practically if compression is used.

Preferably, immersion is used to achieve highest possible sensitivity of the imaging. With immersion, all portions of the breast are imaged, with nearly the same illumination reaching the detector and providing more optimal use of the dynamic range of the camera. That is, the entire image may be acquired with a high level of illumination, and hence high SNR. For the non-immersed breast, variations in the transmitted light intensity across the breast will be large. To avoid camera saturation in the thinnest regions, low light levels will be obtained in the thicker regions. Thus, the thicker regions will have a lower SNR, and worse imaging results. Researchers have used the phase measurement available with frequency domain measurements to perform correction for edge effects. Immersion achieves a similar goal.

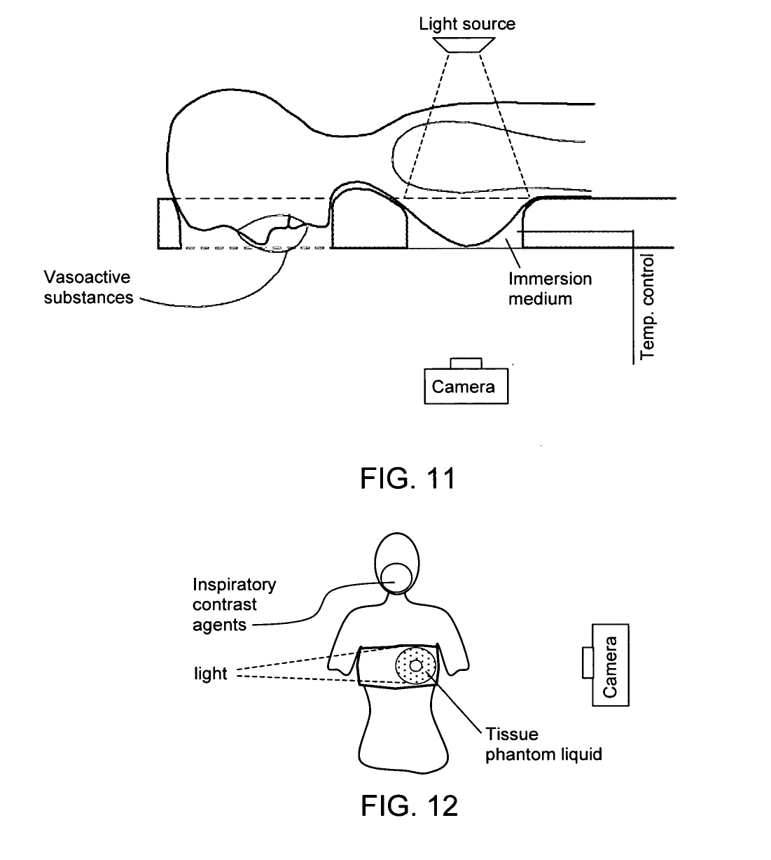

Immersion can be achieved in at least two ways as shown in FIGS. 11 and 12. In FIG. 11, a human subject lies prone on a table similar to a stereotactic breast biopsy table with the breast immersed in a matching medium below. The light does not have to pass through the entire human torso. The optical measurements can be made with the light passing through the region of interest only. In this example, the light source illuminates across the breast only and not the entire torso. Preferably, the subject is provided with one or more premixed gas mixtures containing vasoactive substances/agents, such as oxygen and carbon dioxide, by any method and apparatus that conveniently and comfortably deliver the gas to be inhaled. The system set up in both FIGS. 11 and 12 is similar to those shown in FIG. 1(a) and FIG. 1(b), although the system set up shown in FIG. 11 can also be used without immersion.

In FIG. 12, the breast is surrounded with a doughnut-shaped transparent bag containing a tissue phantom liquid. The bag would be filled to a slight overpressure to press against the breast in a manner similar to a blood pressure cuff, except that the overpressure would be much less. This method would achieve the same advantage of immersion but with less preparation and cleanup required. Preferably, the second immersion method is employed where a new bag with fresh immersion medium is used for each human subject. The immersion medium should be maintained at 37° C.

Where possible, optical imaging is preferably performed before any biopsy procedure. This avoids any influence the biopsy procedure might have on imaging measurement and interpretation. The imaging may be performed using only one or two inhalation protocols so that the total imaging takes only a few minutes.

As one skilled in the art will appreciate, the relative sensitivity and specificity of a diagnostic method depend on the criteria used. Relevant criteria include percentage change in hemoglobin content and hemoglobin oxygenation, and the relative signs (i.e., did each increase or decrease). By varying the criteria used either sensitivity or specificity can be made high, but at the expense of the other dimension. To assist in the analysis of the data, we use a receiver operating characteristic (ROC) curve, which plots sensitivity versus false positive fraction; the free parameter is the criterion or threshold used for diagnosis. The area under the ROC curve gives a measure of the quality of the method; an area near 1 is desirable. The ROC curves are prepared for each contrast mechanism and for the contrast mechanisms in conjunction.

Gas Protocols

Because of the different respiratory rate, heart rate, size, and the fact that the animals used in animal models of the invention are anesthetized and humans would not be, gas protocols are different for humans and animals. Measurements are performed on animals and/or humans with varying inhalation gas composition and administration time to establish proper protocols for gas inhalation.

In the examples disclosed herein, gas mixtures of air, O₂, CO₂, and O₂+CO₂ are produced on demand using computer-controlled gas flow controllers. In some embodiments, two gases are used: O₂ and CO₂. In some embodiment, three gases are used to produce these mixtures: nitrogen, O₂, and CO₂. In some cases, mixtures of these gases may be prepared at fixed mixture ratios, and the gas inhalation protocol would involve switching between breathing of the premixed gases.

The gas flow controllers can rapidly alternate among gas compositions, continuously varying the levels of CO₂ and O₂ in, for example, a nitrogen buffer, or create carbogen. Because CO₂ and O₂ have opposing effects on vasculature (vasodilation versus vasoconstriction, respectively), using these two mechanisms in opposition or in alternation should produce useful results from the differential vasoactive imaging. For example, elevated CO₂ levels may be administered for a period of one minute, followed rapidly by a period of elevated O₂. The same protocol could be repeated with a small overlap between the elevated CO₂ and O₂ levels.

Carbon dioxide is toxic when administered at high concentrations and carbon dioxide levels must be maintained at levels of 5% or less to avoid such toxicity. The literature indicates that concentrations as low as 2% achieve practical vascular activity for radiotherapy with good patient tolerance, see, e.g., incorporated herein by reference, H. Baddeley, P. M. Brodrick, N. J. Taylor, M. O. Abdelatti, L. C. Jordan, A. S. Vasudevan, H. Phillips, M. I. Saunders, and P. J. Hoskin, “Gas exchange parameters in radiotherapy patients during breathing of 2%, 3.5% and 5% carbogen gas mixtures,” Br. J. Radiol. 73, 1100-1104 (2000). In the present invention, carbon dioxide levels are preferably 0% to 5%.

With the computer-controlled flow controllers, we can sequentially administer different gas mixtures, which may or may not be premixed, to the same individual, taking care that the vasculature recovers sufficiently between the changes. We expect more effective discrimination between cancerous and noncancerous tissue, which ultimately may be utilized as a means of distinguishing among different tumor types.

Image Analysis Tools

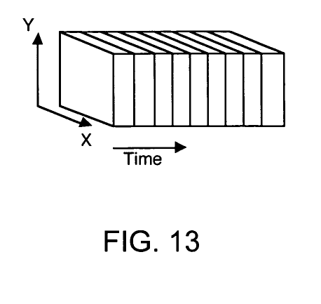

The measurements acquired for differential vasoactive imaging comprise three-dimensional (3-D) datasets as illustrated in FIG. 13. The two spatial dimensions and one temporal dimension differ from other 3-D imaging modalities such as MRI or computed tomography (CT), which have three spatial dimensions. For those imaging modalities, visualization tools often create 2-D images as cross sections through the 3-D data set. Regions of interest can be probed by changing the orientation of the cross section. This is similar to an ultrasound technician changing the orientation of the ultrasound probe.

Visualization of data such as in FIG. 13 can be performed by taking cross sections at different orientations. FIGS. 2-4 are examples of a cross section and a line section through such a data set at constant time and position, respectively. However, both the spatial pattern (such as FIG. 2(b)) and the temporal pattern (such as FIG. 3) are necessary to define features in this data set. Simultaneously capturing both of these features requires a different sort of image analysis tool. One such tool is PCA. Applying PCA, the DVOI approach can be readily adapted to allow automated data processing of temporal image data sets of oxyhemoglobin, deoxyhemoglobin, and total hemoglobin, and change in O₂ content.

To improve the results, the DVOI approach may also adapt methods such as spatial and temporal averaging conditioned on the image features and the use of a priori information such as the temporal profile of the gas inhalation protocol. For breast imaging, the number of eigen images may be larger. The DVOI approach may therefore adapt methods for classifying the eigen images (e.g., by tumor type, other feature such as blood vessels).

Enhancing the Differential Vasoactive Optical Imaging System

As one skilled in the art will appreciate, the DVOI system disclosed herein can be optimized or otherwise modified to improve its performance by, for example, adding another wavelength to enhance the imaging of water, increasing the illumination power, and increasing camera sensitivity. These modifications can enable imaging through large tissue phantoms with SNR (signal to noise ratio) limited only by shot noise, which is a fundamental limitation for any imaging process. High SNR can be very effective for differential imaging because image heterogeneity is removed during the image subtraction process. That is, subtraction of two images taken of the same field of view yields an image of zero intensity if nothing has changed.

1. Enhance Imaging of Water

Water concentrations are known to influence measurements of hemoglobin. Thus, performing imaging at a wavelength dominated by water absorption should assist in quantifying oxyhemoglobin and deoxyhemoglobin measurements. Because of the high fraction of water in blood, images with dominant water absorption should also help monitor blood volume directly. Although the change in water content associated with vasodilation or vasoconstriction is relatively small, we have found that the differential imaging is quite sensitive to such changes. Thus, it is possible to monitor changes in blood volume directly using differential images at 970 nm, a wavelength dominated by water absorption. A water-based measurement of blood volume can also provide information on blood plasma changes, which are somewhat different from the changes provided by hemoglobin measurements. Measuring blood plasma and/or monitoring blood volume changes with water absorption are not critical to the success of our imaging approach, but they potentially could make the overall imaging approach more powerful.

2. Increase Illumination

The images shown with reference to the Working Examples section were obtained using 21 LEDs at each wavelength. The power available from the LED array can be increased by a factor of 20 with more LEDs. Their brightness can also be increased by operating them at higher drive currents. Burn-in tests showed that the LEDs can be operated significantly above their typical operating currents for many weeks without incurring problems. The LEDs are turned on for only short periods during imaging at each wavelength, thereby increasing the practicality of higher current operation without LED damage.

3. Increase Camera Sensitivity

The camera sensitivity can be readily increased with a more sensitive camera such as a Retiga EXi camera produced by Q-Imaging. This CCD camera is approximately two times more sensitive in the NIR than the one used in the Examples above. In addition, the camera-sensitive area is four times larger. These two improvements will lead to an overall enhancement in camera sensitivity of roughly a factor of 8. In combination, the increased illumination and more sensitive camera should improve overall system sensitivity by more than 100 times.

Although the present invention and its advantages have been described in detail, it should be understood that the present invention is not limited to or defined by what is shown or described herein. Known methods, systems, or components may be discussed without giving details, so to avoid obscuring the principles of the invention. As it will be appreciated by one of ordinary skill in the art, various changes, substitutions, and alternations could be made or otherwise implemented without departing from the principles of the present invention. Accordingly, examples and drawings disclosed herein are for purposes of illustrating a preferred embodiment(s) of the present invention and are not to be construed as limiting the present invention. Rather, the scope of the present invention should be determined by the following claims and their legal equivalents.

1. A method of imaging a region of interest, comprising:
acquiring images through said region of interest;
introducing varying levels of inspiratory contrast agents to said region of interest, said inspiratory contrast agents stimulating vascular changes in said region of interest; and
obtaining optical measurements on oxy- and deoxy-hemoglobin of said region of interest during said introducing step, thereby acquiring differential vascular function information useful in detecting cancerous tumors.

2. The method according to claim 1, further comprising the step of:
positioning said region of interest between a light source and a camera.

3. The method according to claim 1, further comprising the step of:
immersing said region of interest in a matching medium.

4. The method according to claim 1, further comprising the step of:
maintaining said matching medium at 37° C.

5. The method according to claim 1, further comprising the step of:
mildly compressing said region of interest.

6. The method according to claim 1, wherein
said inspiratory contrast agents are oxygen and carbon dioxide.

7. The method according to claim 1, wherein said region of interest is a breast of a human subject.

8. The method according to claim 5, further comprising the step of:
administering, by inhalation, said human subject with a gas mixture composed of air and said inspiratory contrast agents, wherein said inspiratory contrast agents are oxygen and carbon dioxide.

9. The method according to claim 1, further comprising the step of:
automatically controlling said varying levels with one or more flow controllers.

10. A system configured to implement the method steps of claim 1.

11. A noninvasive method of detecting cancerous tumors in vivo, comprising the steps of:
utilizing differential vasoactive optical imaging to acquire images through a region of interest before and during inhalation of varying levels of vasoactive agents; wherein
said vasoactive agents are oxygen and carbon dioxide; and wherein
said vasoactive agents stimulate vascular changes in said region of interest, resulting dramatically increase in contrast between cancerous and noncancerous tissue in said region of interest.

12. The method according to claim 11, wherein said region of interest is an optically accessible area of a human body.

13. The method according to claim 11, wherein said region of interest is a human breast.

14. An imaging system comprising:
a means for administering varying levels of vasoactive agents to a human or animal subject having a region of interest;
a near infrared light source directed at said region of interest;
an image acquisition means for acquiring images of said region of interest before and during administration of said vasoactive agents; and
a processing means for analyzing said images to identify vasculature associated with angiogenic vasculature in cancerous tumors.

15. The imaging system of claim 14, wherein
said vasoactive agents are oxygen and carbon dioxide.

16. The imaging system of claim 14, wherein
said image acquisition means is a charge-coupled device camera that is sensitive in near infrared.

17. The imaging system of claim 14, wherein
said near infrared light source is an array of light emitting diodes capable of operating at a plurality of wavelengths including 780 nm, 840 nm and 970 nm.

18. The imaging system of claim 14, further comprising:
an immersion medium immersing said region of interest; and
a holding means containing said immersion medium.

19. The imaging system of claim 18, wherein
said immersion medium is a tissue phantom liquid having optical properties substantially matching those of said region of interest.

20. The imaging system of claim 18, wherein
said holding means is a doughnut-shaped transparent bag filed to a slight overpressure to press against said region of interest.

21. The imaging system of claim 14, further comprising:
one or more flow controllers for controlling levels of said vasoactive agents being administered to said subject.

22. The imaging system of claim 21, wherein
said flow controllers are capable of rapidly alternating among different gas compositions containing said vasoactive agents while continuously varying levels of said vasoactive agents.
